(19) United States
(12) Patent Application Publication
(10) Pub. No.: US 2023/0408531 A1
(43) Pub. Date: Dec. 21, 2023

(54) USE OF MALIC ENZYME 2 IN PREPARATION OF DIAGNOSTIC REAGENT OR MEDICAMENT FOR SILICOSIS OR PULMONARY FIBROSIS-RELATED DISEASE

(71) Applicant: Institute of Basic Medical Sciences of Chinese Academy of Medical Sciences, Beijing (CN)

(72) Inventors: Jing WANG, Beijing (CN); Tiantian ZHANG, Beijing (CN); Youliang SUN, Beijing (CN); Xianmei QI, Beijing (CN)

(73) Assignee: Institute of Basic Medical Sciences of Chinese Academy of Medical Sciences, Beijing (CN)

(21) Appl. No.: 17/929,831

(22) Filed: Sep. 6, 2022

(30) Foreign Application Priority Data
Jun. 21, 2022 (CN) 202210703092.4

(51) Int. Cl. G01N 33/68; C12Q 1/32

(52) U.S. Cl. CPC G01N 33/6893; C12Q 1/32; G01N 2800/12; C12Q 2600/158; A01K 67/0276

(57) ABSTRACT

The present disclosure provides use of malic enzyme 2 (ME2) in preparation of a diagnostic reagent or a medicament for silicosis or pulmonary fibrosis-related diseases, and belongs to the technical fields of medical treatment and medicine. Research results of the present disclosure show that ME2 knockout significantly alleviates inflammatory response and fibrotic lesions in mice with silicosis. Based on the above research results, the present disclosure provides use of ME2 in treatment of pulmonary inflammatory responses and pulmonary fibrotic lesions of silicosis or pulmonary fibrosis-related diseases. Expression of ME2 is inhibited to alleviate the inflammatory response and fibrotic lesions of the silicosis, providing support for exploring a targeted drug for treating pulmonary inflammatory responses and pulmonary fibrosis of silicosis or pulmonary fibrosis-related diseases.

### CROSS REFERENCE TO RELATED APPLICATION

This patent application claims the benefit and priority of Chinese Patent Application No. 202210703092.4, filed on Jun. 21, 2022, the disclosure of which is incorporated by reference herein in its entirety as part of the present application.

### TECHNICAL FIELD

The present disclosure relates to the technical fields of medical treatment and medicine, in particular to use of malic enzyme 2 (ME2) in preparation of a diagnostic reagent or a medicament for silicosis or pulmonary fibrosis-related diseases.

### BACKGROUND ART

Pneumoconiosis a group of lung diseases caused by chronically breathing in different types of pathogenic dust, which is a world's main occupational disease manifesting as diffuse pulmonary fibrosis. Late pneumoconiosis can cause serious pulmonary dysfunction. According to different types of dust that is breathed in, pneumoconiosis can be divided into coal worker's pneumoconiosis, asbestosis, silicosis, and other categories. Silicosis is one of the most common subgroups in pneumoconiosis and is a type of pulmonary fibrosis caused by chronically inhaling free silica crystals and retaining in lungs. Its main pathological features are diffuse interstitial fibrosis and siliconic nodule formation. Silica in industrial dust has the strongest ability to induce pulmonary fibrosis, and silicosis induced by silica stimulation is the most severe in pneumoconiosis. In recent years, because introduction of silica in emerging industries is poorly understood and fails to control, the global incidence of pneumoconiosis, particularly silicosis, is rising year by year.

The pathogenesis and development of silicosis go through two stages: chronic inflammatory response and fibrosis progression. The inhalation and retention of silica dust may promote inflammatory responses in inflammatory cells of lung tissue, release multiple types of cytokines, and further accelerate fibroblast proliferation and develop pulmonary fibrosis. Herein, macrophages in the lung tissue play a dominant role. Silica is first recognized and phagocytized by the surface receptor of the alveolar macrophage (scavenger receptor) after it enters the lungs, and the phagocytized silica crystal is fused with intracellular lysosome to form a phagosome. Silica crystal cannot be digested, which can lead to abnormal autolysosome system of the alveolar macrophage and further promote chronic inflammatory responses and fibrosis progression.

Recruitment of neutrophils and lymphocytes in the lung tissue may aggravate inflammatory responses and promote subsequent fibrosis progression. Cytokines such as interleukin-6 (IL-6), interleukin-1β (IL-1β), tumor necrosis factor-α (TNF-α), and transforming growth factor β (TGF-β) participate in inflammatory responses and fibrosis progression. Pulmonary fibrosis seriously affects patients' respiratory functions and may develop into pulmonary dysfunction at the late stage. The existing therapeutic drugs can alleviate pulmonary inflammatory responses and delay the progression time from inflammatory response to fibrosis stage in the lung tissue, but pulmonary fibrosis cannot be reversed in patients. Therefore, it is still urgent to screen out a target for effectively improving pulmonary fibrosis to treat pulmonary fibrosis.

A growing body of research has revealed that metabolic reprogramming can influence fibrosis and other non-neoplastic diseases (for example, idiopathic pulmonary fibrosis (IPF)), but a considerable portion of mechanisms overlap between pulmonary fibrosis and cancer. Herein, some common metabolic characteristics are included, for example, increased glycolytic rate, high expression of glycolytic enzymes, and enhanced serine-glycine de novo synthesis. In addition, metabolic reprogramming can influence macrophage function and thus inflammatory responses, for example, succinic acid, an intermediate metabolite in the tricarboxylic acid cycle, can respond to lipopolysaccharide (LPS) stimulation and promote macrophage M1 polarization. Alpha ketoglutarate (α-KG), an important metabolite in the tricarboxylic acid cycle, can inhibit hypoxia-inducible factor-1α (HIF-1α) and interleukin-1β (IL-1β) in M2 macrophages.

Malic enzyme (ME) is a key enzyme that regulates malic acid metabolism in the tricarboxylic acid cycle, which is a reversible reaction that catalyzes oxidative decarboxylation of malic acid into pyruvic acid and accompanies the production of nicotinamide adenine dinucleotide phosphate (NADPH). So far, three subtypes of ME have been identified in mammals, which are encoded by three homologous genes, respectively. According to their cellular distribution and coenzyme specificity, they were named cytoplasmic NADP-dependent ME (ME1), mitochondrial NAD(P)-dependent ME (ME2), and mitochondrial NADP-dependent ME (ME3), respectively, of which ME1 and ME2 are main subtypes. Mitochondrial NAD(+)-dependent malic enzyme 2 (ME2) can catalyze malic acid to yield pyruvic acid and CO₂, reduce NAD(+) into NADH, and regulate redox equilibrium reaction, cellular energy metabolism, and biosynthesis of molecules. ME2 is significantly highly expressed in a plurality of cancers. A plurality of studies indicate that it can be used as a novel biomarker for cancer diagnosis and a therapeutic drug target; targeting ME2 can significantly inhibit the proliferation, migration, and invasion of tumor cells. Experimental results show that ME2 is significantly highly expressed in fibrotic lung tissue caused by silicosis, but the function of ME2 in pulmonary fibrosis remains unclear.

### SUMMARY

In view of this, an objective of the present disclosure is to provide use of ME2 in preparation of a diagnostic reagent or a medicament for silicosis or pulmonary fibrosis-related diseases, providing support for exploring a targeted drug for treating pulmonary inflammatory responses and pulmonary fibrosis of pulmonary fibrosis-related diseases.

To achieve the above objective, the present disclosure provides the following technical solution:

Use of ME2 in preparation of a diagnostic reagent or a medicament for silicosis or pulmonary fibrosis-related diseases is provided.

Preferably, the ME2 may be used as a biomarker for screening the silicosis or the pulmonary fibrosis-related diseases in preparation of related products for diagnosing or treating the silicosis or the pulmonary fibrosis-related diseases.

Preferably, the silicosis or the pulmonary fibrosis-related diseases may include pulmonary inflammatory response of silicosis and pulmonary fibrosis.

Preferably, the pulmonary inflammatory response and the fibrosis may be diagnosed by detection of an expression level of the ME2 in a lung tissue.

More preferably, the detection may include mRNA and/or protein levels of the ME2.

Preferably, ME2 gene in a macrophage may be knocked out to down-regulate levels of inflammatory factors in lungs.

Preferably, the ME2 gene in the macrophage may be knocked out to reduce hydroxyproline content in the lung tissue and degree of pulmonary fibrosis.

Compared with the prior art, the present disclosure has the following beneficial effects:

The present disclosure provides use of ME2 in preparation of a diagnostic reagent or a medicament for silicosis or pulmonary fibrosis-related diseases. Research results of the present disclosure show that ME2 knockout significantly alleviates inflammatory response and fibrotic lesions in mice with silicosis. Based on the above research results, the present disclosure provides use of ME2 in treatment of pulmonary inflammatory responses and pulmonary fibrotic lesions of silicosis or pulmonary fibrosis-related diseases. Expression of ME2 is inhibited to alleviate inflammatory responses and fibrotic lesions of the silicosis or the pulmonary fibrosis-related diseases, providing support for exploring a targeted drug for treating pulmonary inflammatory responses and pulmonary fibrosis of silicosis or pulmonary fibrosis-related diseases.

### BRIEF DESCRIPTION OF THE DRAWINGS

FIG. 1 is a schematic diagram of experimental results of levels of ME2 in lung tissues of pneumoconiosis patients. FIG. 1A illustrates a real-time qPCR assay result; FIG. 1B illustrates a Western blot result. ACTIN serves as an internal reference for real-time qPCR assay and Western blot.

FIG. 2 is a schematic diagram of experimental results of levels of ME2 in lung tissues of pneumoconiosis model mice. FIG. 2A illustrates a real-time qPCR assay result; FIG. 2B illustrates a Western blot result. ACTIN serves as an internal reference for real-time qPCR assay and Western blot.

FIG. 3 is a schematic diagram of experimental results of cellular localization of ME2 in lung tissues of pneumoconiosis patients and pneumoconiosis model mice; FIG. 3A and FIG. 3B illustrate result analyses of single-cell sequencing; FIG. 3C illustrates immunofluorescence staining results of lung tissue sections of patients with pneumoconiosis; FIG. 3D illustrates immunofluorescence staining results of lung tissue sections of silicosis model mice.

FIG. 4 is a schematic diagram of experimental results of mRNA expression levels of inflammatory factors in lung tissues of pneumoconiosis model mice. FIG. 4A illustrates expression levels of IL-1β mRNA in mouse lung tissues; FIG. 4B illustrates expression levels of IL-6 mRNA in mouse lung tissues; FIG. 4C illustrates expression levels of TNF-α mRNA in mouse lung tissues. ME2^{F/F} represents macrophage conditional knockout control mice, and ME2^{F/F}/Lyz2^{Cre} represents macrophage conditional ME2 knockout mice (n=12, *, P<0.05, **, P<0.01, ***, P<0.001, ****, P<0.0001). Actin serves as an internal reference for real-time qPCR assay.

FIG. 5 is a schematic diagram of experimental results of levels of inflammatory factors in bronchoalveolar lavage fluids (BALF) of pneumoconiosis model mice. FIG. 5A illustrates expression levels of IL-1β in mouse BALF; FIG. 5B illustrates expression levels of IL-6 in mouse BALF; FIG. 5C illustrates expression levels of TNF-α in mouse BALF. ME2^{F/F} represents macrophage conditional knockout control mice, and ME2^{F/F}/Lyz2^{Cre} represents macrophage conditional ME2 knockout mice (n=12, *, P<0.05, **, P<0.01, ***, P<0.001, ****, P<0.0001).

FIG. 6 illustrates experimental results of inflammatory responses and collagen accumulation in lung tissues of pneumoconiosis model mice. FIG. 6A illustrates hematoxylin-eosin (HE) staining results; FIG. 6B illustrates inflammatory response scoring results; FIG. 6C illustrates Masson staining results; FIG. 6D illustrates fibrosis scoring results. ME2^{F/F} represents macrophage conditional knockout control mice, and ME2^{F/F}/Lyz2^{Cre} represents macrophage conditional ME2 knockout mice (n=12, *, P<0.05, **, P<0.01, ***, P<0.001, ****, P<0.0001).

FIG. 7 is a schematic diagram of experimental results of expression levels of fibrotic marker Col1a1 in lung tissues of pneumoconiosis model mice. FIG. 7A illustrates expression results of Col1a1 mRNA in mouse lung tissues, and Actin serves as an internal reference for real-time qPCR assay; FIG. 7B illustrates statistics of the positive area of Col1a1 in mouse lung tissues. FIG. 7C illustrates immunohistochemistry (IHC) of Col1a1 mouse lung tissues. ME2^{F/F} represents macrophage conditional knockout control mice, and ME2^{F/F}/Lyz2^{Cre} represents macrophage conditional ME2 knockout mice (n=12, *, P<0.05, **, P<0.01, ***, P<0.001, ****, P <0.0001).

FIG. 8 is a schematic diagram of expression results of fibronectin in lung tissues of pneumoconiosis model mice. FIG. 8A illustrates experimental results of fibronectin mRNA levels; FIG. 8B illustrates experimental results of fibronectin protein levels; FIG. 8C illustrates statistics of expression of fibronectin protein. ME2^{F/F} represents macrophage conditional knockout control mice, and ME2^{F/F}/Lyz2^{Cre} represents macrophage conditional ME2 knockout mice (n=12, *, P<**, P<0.01, ***, P<0.001, ****, P<0.0001). Actin serves as an internal reference for Western blot.

FIG. 9 is a schematic diagram of detection results of hydroxyproline in lung tissues of pneumoconiosis model mice. ME2^{F/F} represents macrophage conditional knockout control mice, and ME2^{F/F}/Lyz2^{Cre} represents macrophage conditional ME2 knockout mice (n=12, *, P<0.05, **,P<0.01,***, P<0.001,****,P<0.0001).

FIG. 10A-E illustrates pathological sections of lung tissues of five pneumoconiosis patients.

### DETAILED DESCRIPTION OF THE EMBODIMENTS

The technical solution provided by the present disclosure will be described in detail below with reference to examples, but they should not be construed as limiting the protection scope of the present disclosure.

### EXAMPLE 1

ME2 was significantly expressed in lung tissues of pneumoconiosis patients and mouse models.

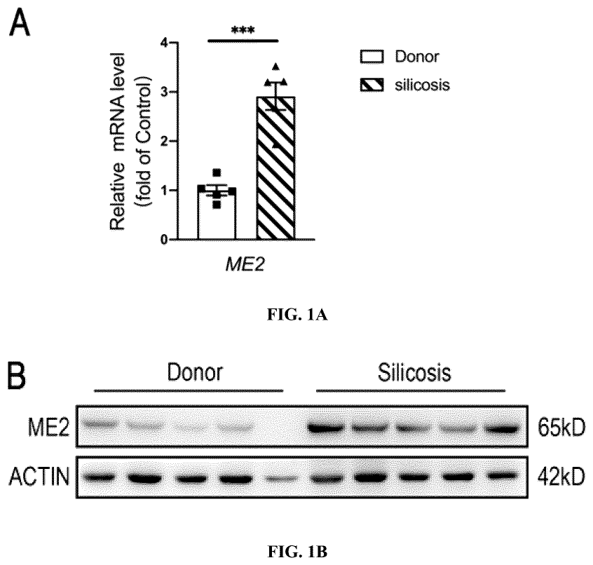

In the present disclosure, expression levels of ME2 were detected by qPCR and Western blot after proteins and RNAs were extracted from lung tissues collected from five normal volunteers and five pneumoconiosis patients. Results found that levels of ME2 mRNA and protein in lung tissues of pneumoconiosis patients were significantly upregulated compared with normal volunteers (FIG. 1). Samples in the present disclosure were from lung transplant samples from pneumoconiosis patients of the China-Japan Friendship Hospital. The present study was approved by the Institutional Review Board of the Institute of Basic Medical Sciences, Chinese Academy of Medical Sciences (Project No.: 062-2019). All subjects signed informed consent forms.

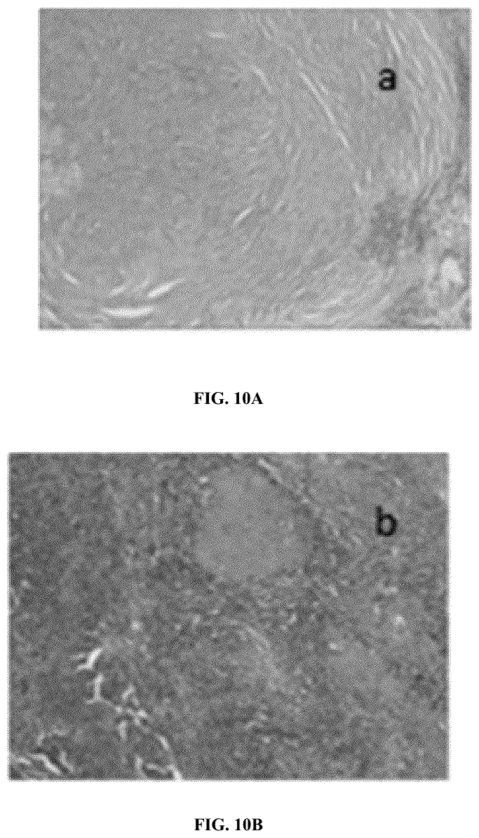

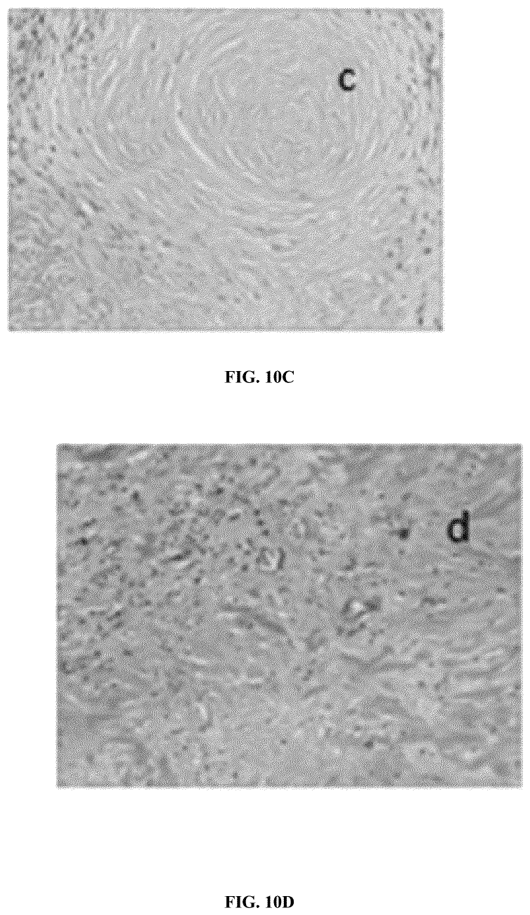

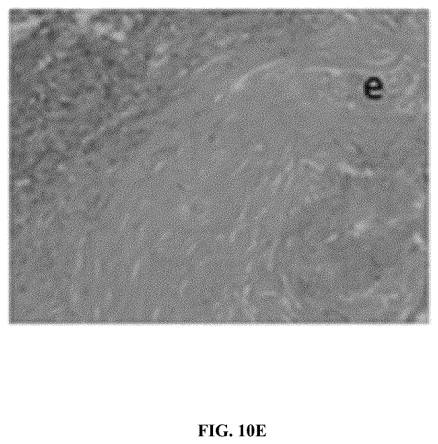

TABLE 1
Pathological sections of lung tissues
of five pneumoconiosis patients

| | | | Pathological |
|---|---|---|---|
| Patient | Sex | Medical history | section |
| 1 | Male | Pneumoconiosis, interstitial lung disease, | FIG. 10A |
| | | and gastroesophageal reflux | |
| 2 | Male | Pneumoconiosis, and history of pulmonary | FIG. 10B |
| | | tuberculosis | |
| 3 | Male | Silicosis | FIG. 10C |
| 4 | Male | Interstitial lung disease Previous history | FIG. 10D |
| | | of contact with coal for 10 years | |
| 5 | Male | Interstitial lung disease, history of | FIG. 10E |
| | | pulmonary tuberculosis, and working | |
| | | in a coal mine for more than 10 years | |

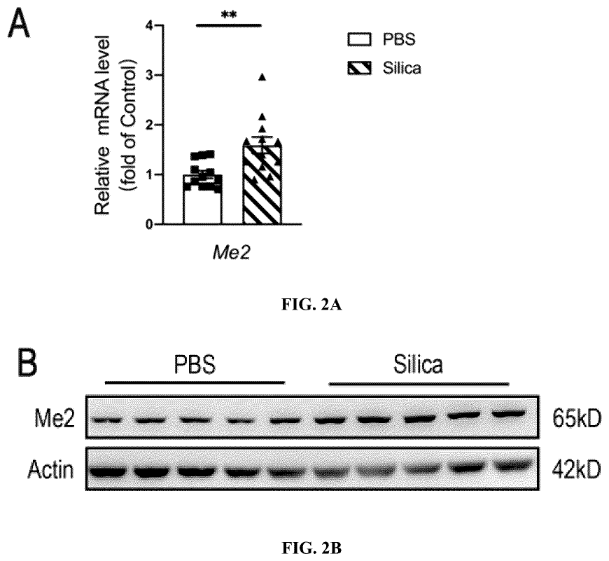

To further determine the change in expression of ME2 in pneumoconiosis, the mice were modeled by single-dose intratracheal instillation of 600 mg/kg silica; after six weeks, modeling was completed, mouse lung tissues were collected, and expression levels of ME2 mRNA and protein in mouse lung tissues were detected by qPCR and Western blot. Results showed that compared with the control group (PBS), levels of both ME2 mRNA and protein in lung tissues of silicosis model mice (FIG. 2). This result was consistent with the trend of change in expression of ME2 in lung tissues of the above pneumoconiosis patients.

### EXAMPLE 2

ME2 was mainly expressed in macrophages of lung tissues.

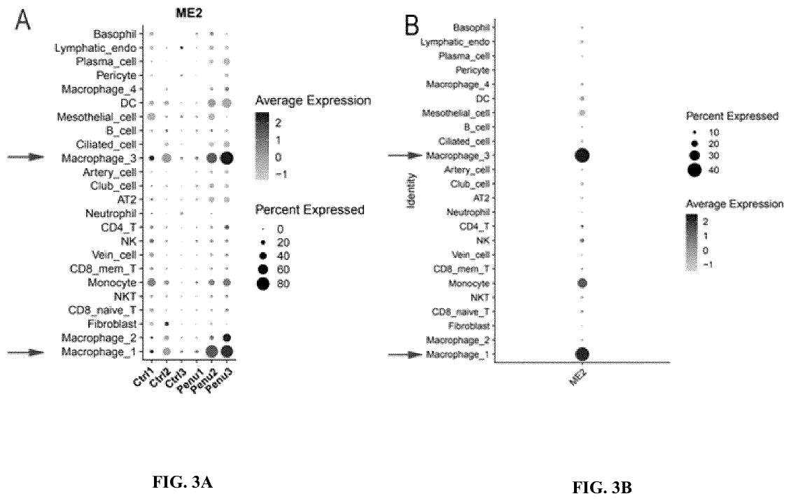

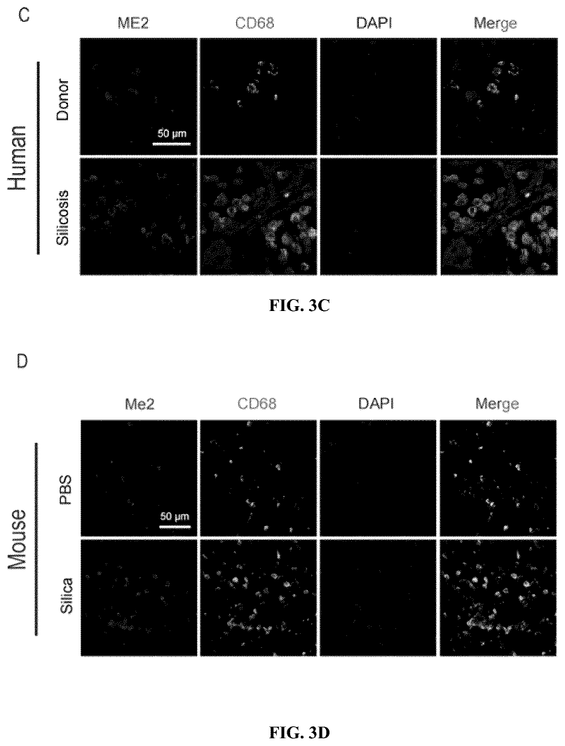

The lung is a heterogeneous organ orderly composed of a plurality of types of cells. To further explore an effector cell where ME2 serves a function, the present disclosure found from the analysis of single cell transcriptome data of lung tissues of silicosis model mice that the expression of ME2 was significantly upregulated after silica stimulation, and the ME2 was mainly present in macrophages (FIGS. 3A and 3B). Subsequently, verification was conducted by immunofluorescence co-localization. Results found that ME2 was highly expressed in macrophages of lung tissues of both pneumoconiosis patients and model mice (FIGS. 3C and 3D). Macrophage is the most important effector cell known during the pathogenesis and development of silicosis. Biological processes such as its phagocytosis of dust, damage of lysosome, and change of cell metabolism level may activate downstream immune inflammatory response and fibrosis pathway. During the progression of silicosis, upregulation of ME2 can promote disease progression by altering macrophage functions.

### EXAMPLE 3

Macrophage conditional ME2 knockout significantly relieved the secretion of inflammatory factors and the inflammatory cells infiltration in lung tissues of pneumoconiosis model mice.

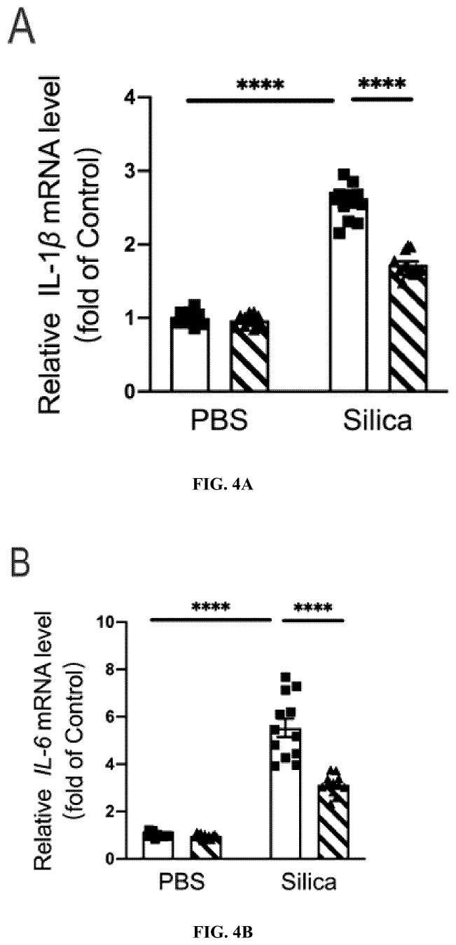

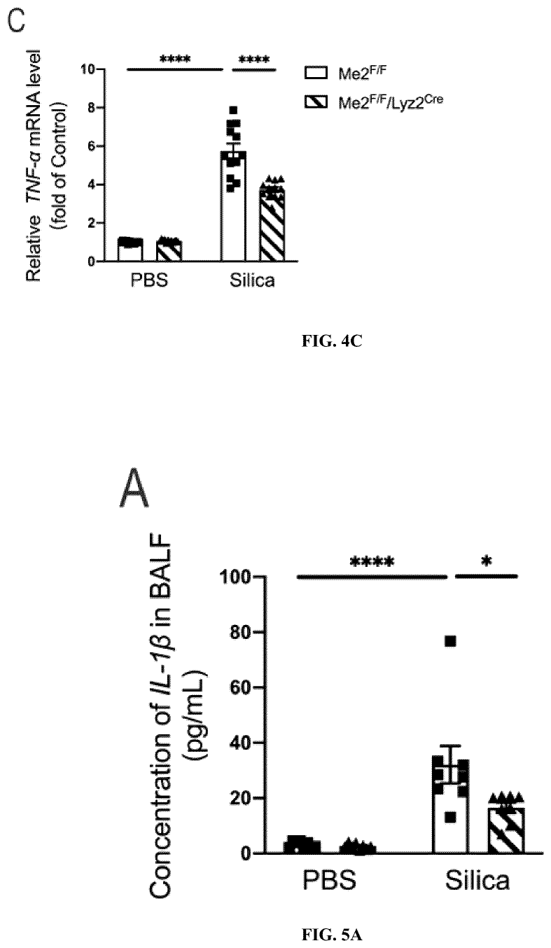

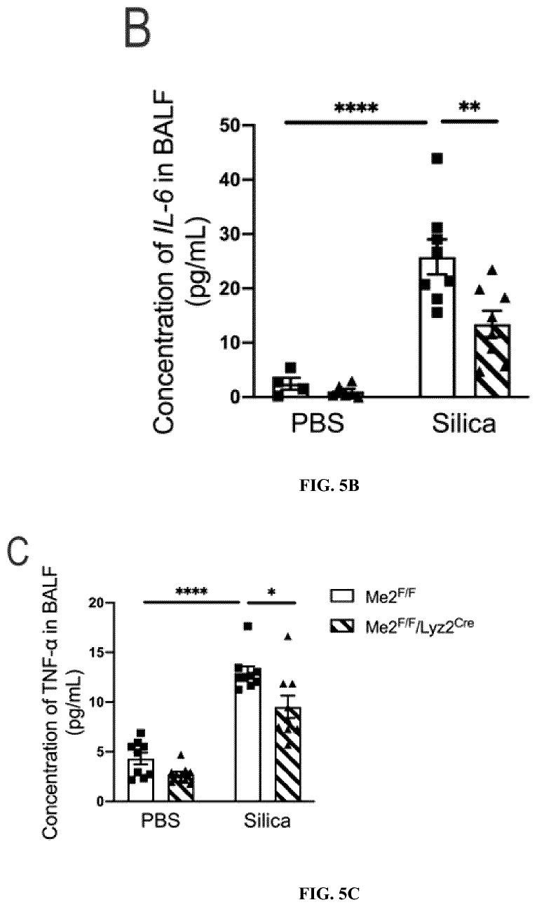

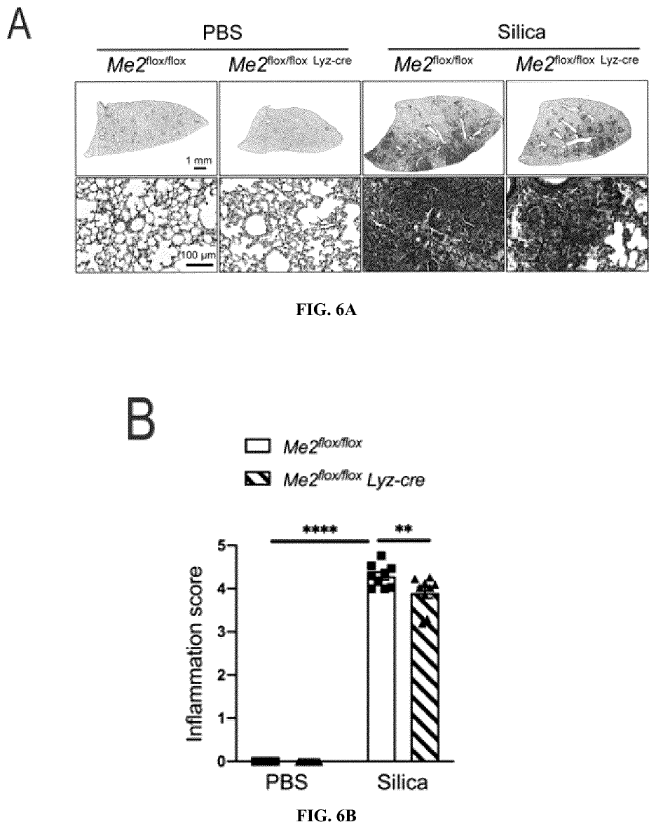

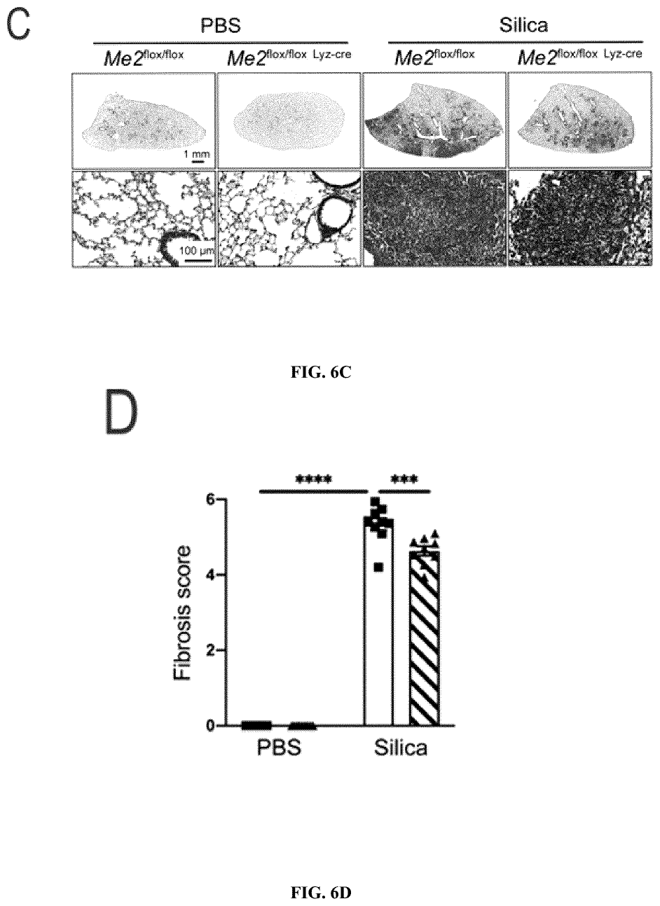

To further reveal the function of the ME2 highly expressed in macrophages in pneumoconiosis, the present disclosure used macrophage conditional ME2 knockout mice to construct a model of pneumoconiosis; lung tissues and BALF were collected from normal mice and model mice, mRNA expression levels of inflammatory factors IL-1β, IL-6, and TNF-α in mouse lung tissues were detected by real-time qPCR (FIG. 4), and expression levels of these three inflammatory factors in BALF were detected by enzyme-linked immunosorbent assay (ELISA) (FIG. 5). In addition, the inflammatory cells infiltration in lung tissues was detected by HE staining (FIGS. 6A and 6B). According to the above three results, it was found that compared with the control group, mRNA and secretion levels of inflammatory factors IL-1β, IL-6, and TNF-α in lung tissues of macrophage conditional ME2 knockout mice were significantly downregulated, and the degree of inflammatory cells infiltration in lung tissues was significantly improved. Thus, it concludes that knockout of highly expressed ME2 in macrophages can reduce the secretion of inflammatory factors in pneumoconiosis lung tissues and relieve pulmonary inflammatory responses.

### EXAMPLE 4

Macrophage conditional ME2 knockout significantly reduced fibrosis levels of lung tissues of pneumoconiosis model mice.

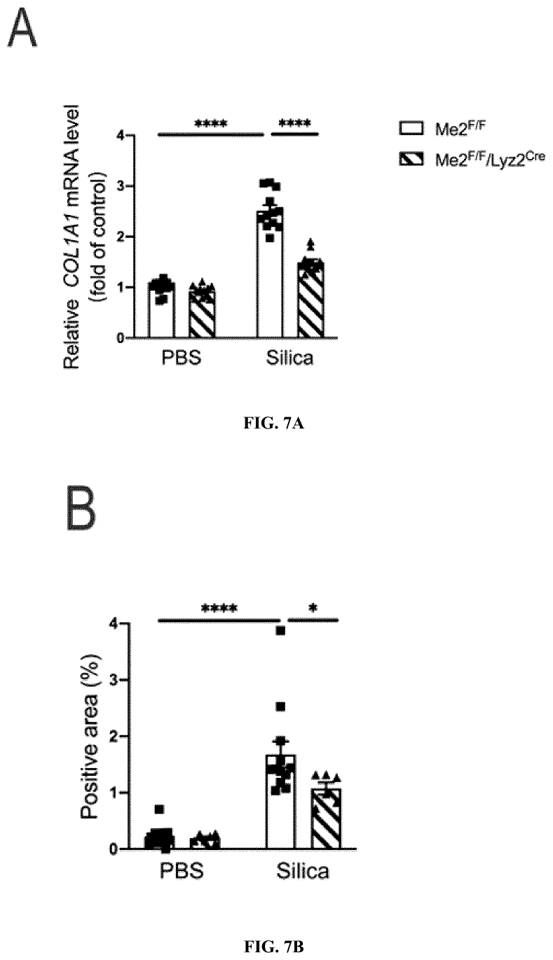

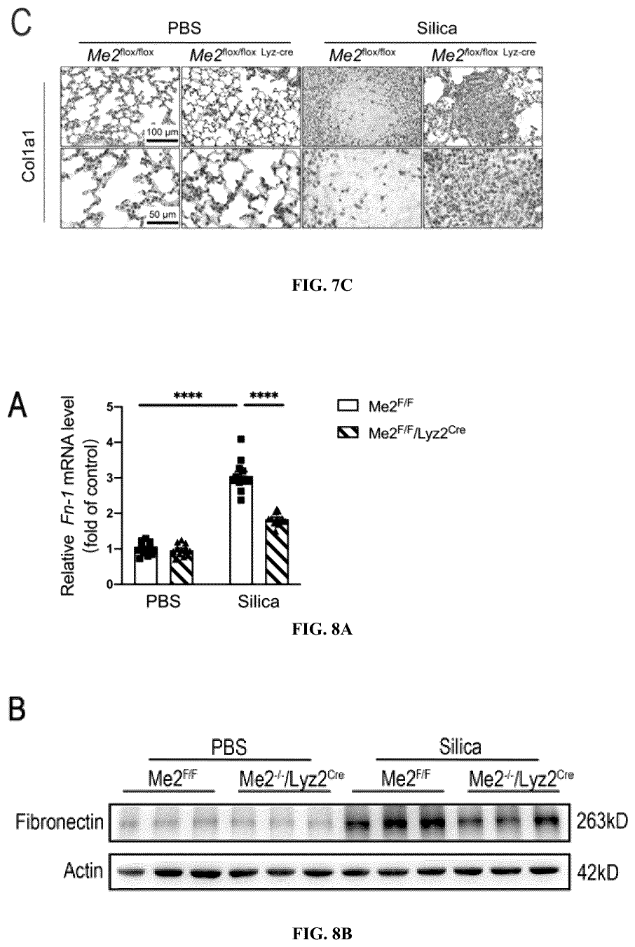

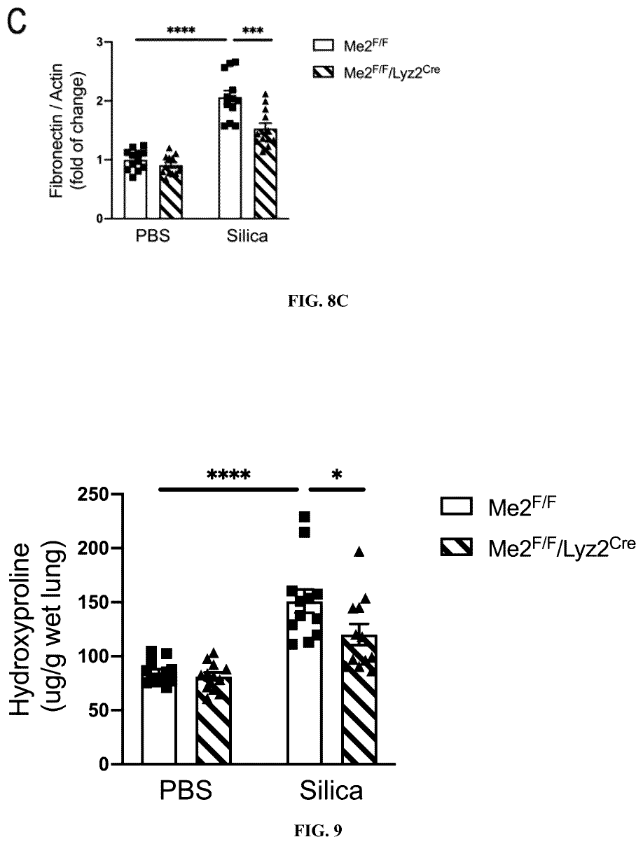

To determine the effect of macrophage conditional ME2 knockout on pulmonary fibrosis, the present disclosure conducted Masson staining on paraffin sections of lung tissues of pneumoconiosis model mice. Results showed that the fibrotic degree of lung tissues of macrophage conditional ME2 knockout mice was significantly reduced (FIGS. 6C and 6D). Meanwhile, the present disclosure used real-time qPCR, IHC, and Western blot to detect mRNA and protein expression levels of pulmonary fibrosis marker proteins collagen I (Col1a1) and fibronectin (Fn-1) in lung tissues of the above pneumoconiosis model mice. It was found that compared with the control group, both mRNA and protein expression levels of Col1a1 and Fn-1 were significantly downregulated in lung tissues of macrophage conditional ME2 knockout pneumoconiosis model mice (FIGS. 7 and 8). In addition, the content of hydroxyproline (HYP) in mouse lung tissues was detected, the collagen accumulation in lung tissues was directly detected, and the degree of pulmonary fibrosis was judged. Results showed that, after macrophage conditional ME2 knockout, the content of HYP in lung tissues of pneumoconiosis model mice was reduced by approximately 20% (FIG. 9, P<0.05). The above results demonstrate that macrophage conditional ME2 knockout can significantly reduce fibrosis levels of lung tissues of pneumoconiosis model mice.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method for preparing a diagnostic reagent or a medicament for silicosis or pulmonary fibrosis-related diseases using malic enzyme 2 (ME2).

2. The method according to claim 1, wherein the ME2 is used as a biomarker for screening the silicosis or the pulmonary fibrosis-related diseases in preparation of related products for diagnosing or treating the silicosis.

3. The method according to claim 1, wherein the silicosis comprises pulmonary inflammatory response of silicosis and pulmonary fibrosis.

4. The method according to claim 1, wherein the pulmonary inflammatory response and the fibrosis are diagnosed by detection of an expression level of the ME2 in a lung tissue.

5. The method according to claim 4, wherein the detection comprises mRNA and/or protein levels of the ME2.

6. The method according to claim 1, wherein ME2 gene in a macrophage is knocked out to down-regulate levels of inflammatory factors in lungs.

7. The method according to claim 1, wherein the ME2 gene in the macrophage is knocked out to reduce hydroxyproline content in the lung tissue and degree of pulmonary fibrosis.
